(19) United States
(12) Patent Application Publication
(10) Pub. No.: US 2017/0311841 A1
(43) Pub. Date: Nov. 2, 2017

(54) MAGNETIC RESONANCE APPARATUS AND OPERATING METHOD THEREFOR

(71) Applicant: Siemens Healthcare GmbH, Erlangen (DE)

(72) Inventor: Eva Rothgang, Schwaig bei Nuernberg (DE)

(73) Assignee: Siemens Healthcare GmbH, Erlangen (DE)

(21) Appl. No.: 15/582,284

(22) Filed: Apr. 28, 2017

(30) Foreign Application Priority Data
May 2, 2016 (DE) 102016207501.2

(51) Int. Cl. A61B 5/055; A61B 5/00

(52) U.S. Cl. CPC A61B 5/0555; A61B 5/74; A61B 5/704; A61B 5/72

(57) ABSTRACT

In a magnetic resonance (MR) apparatus and an operating method wherein the MR apparatus has a scanner with a patient table that can be moved into a patient receiving region of the scanner, the patient table is held in an extended loading position, and three-dimensional scanning data describing the surface of a patient positioned in the loading position on the patient table are recorded with a 3D camera, which has a field of view that covers the patient table in the loading position. For the determination of at least one patient-related recording parameter, the scanning data are evaluated for the subsequent recording MR data.

### BACKGROUND OF THE INVENTION

### Field of the Invention

The invention concerns a method for operating a magnetic resonance (MR) apparatus for recording magnetic resonance data from a patient, wherein the magnetic resonance apparatus has a patient table that can be moved into a patient receiving region of an MR data acquisition scanner of the apparatus, which, for positioning the patient is held in an extended loading position. The invention also concerns a magnetic resonance apparatus for implementing such a method.

### Description of the Prior Art

The quality and, in the medical field, utility for diagnostic purposes of magnetic resonance data, is determined by a number of recording parameters, a large proportion of which have to be selected on a patient-related basis. For example, the receiving region, i.e. the field of view (FoV), should be selected such that the desired field of interest is also actually depicted. Other recording parameters describing the recording geometry, for example the selection of the phase-encoding direction, can also be dependent upon the individual patient. Moreover, in many cases, it is also possible to take account of further properties of the patient, for example the extent of the patient with respect to wrapping artifacts, and the optimization of individual magnetic resonance sequences with respect to fatter or thinner patients.

With present-day magnetic resonance facilities, such parameters that are dependent on the current patient, i.e. patient-related recording parameters, are set manually, for example via a user interface. This takes up valuable time during the workflow and, in many cases, requires extensive understanding of the physics of magnetic resonance imaging to enable the recording parameters to be set as optimally as possible. Therefore, operators of magnetic resonance facilities who are required to set such recording parameters require extremely complex training.

### SUMMARY OF THE INVENTION

An object of the invention is to provide a method and apparatus for setting patient-related recording parameters that optimize the time sequence of a magnetic resonance examination, and that enable the setting to be easily incorporated in the workflow.

This object is achieved in accordance with the invention by a method of the general type described initially but wherein three-dimensional scanning data describing the surface of a patient positioned in the loading position on the patient table are recorded with a 3D camera, the field of view of which covers the patient table in the loading position and, for the determination of at least one patient-related recording parameter, are evaluated for the subsequent recording of the magnetic resonance data.

According to the invention, a 3D camera is employed, i.e. a camera, which, in addition to the usual image data, is also able to record distance information, which, for example, can be assigned to a pixel of the (two-dimensional) camera image. Therefore, such a 3D camera enables a three-dimensional image of a patient positioned on the patient table to be recorded in the form of scanning data, at least as long the patient is not yet obscured by covering items that partially cover the patient, such as local coils and/or blankets. Hence, the scanning data correspond to a type of 3D depth map from which information about the patient can be derived that permits automatic setting of recording parameters for a subsequent magnetic resonance examination. In this case, the 3D camera is arranged outside the patient receiving region, but can be integrated in the workflow during an examination with the magnetic resonance apparatus in a particularly simple way since the patient table has to be moved out of the receiving region in order to place the patient thereupon, i.e. to load the table. This time can be used to record scanning data without this requiring any additional interaction on the part of an operator. However, for this purpose, it is possible to automatically set at least some recording parameters that are dependent on patient information, i.e. specific patient parameters, which not only saves time, but also makes the setting of the recording parameters less complex. This is particularly advantageous for inexperienced operators. The automatic setting of selected recording parameters can also prevent user errors and hence improve the image quality.

Therefore, the scanning data represent the result of a three-dimensional measurement of the patient thus enabling patient information, in particular patient parameters, to be derived therefrom. This information can be used in turn to determine the at least one recording parameter as optimally as possible. It is usual in this case for further examination information also to be included, for example the object of the magnetic resonance examination to be performed. If, for example, it is known that a region of interest in the patient's abdomen is to be examined, the location of the abdomen can be determined from the scanning data and recording parameters, for example those determining the recording region (scanner field of view), can be set appropriately.

It is particularly advantageous for a 3D camera covering the patient table to be situated centrally above the patient table. The 3D camera thus can be positioned such that the entire patient can be seen as far as possible without recording objects and effects that disrupt the scanning data. To this end, a central arrangement relative to the loading position above the middle of the patient table is suitable, since there are generally no disruptive objects in the coverage area and the patient can be seen from above optimally and completely as possible.

The 3D camera used can be a time-of-flight camera and/or a terahertz camera. Time-of-flight cameras are known in the art and measure a phase shift of emitted light pulses in order to obtain distance information for a pixel. For example, such time-of-flight cameras are used in automotive fields. Such 3D cameras using millimeter waves are particularly advantageously able to penetrate certain, for example sheets or blankets arranged on the patient in order to record the surface of the patient lying therebeneath. Such terahertz cameras are, for example, known from usage in so-called “naked scanners”.

It is advantageous for the coordinate system of the 3D camera and the coordinate system of the magnetic resonance apparatus to be registered, or able to be, with one another. If the 3D camera is fixed relative to the magnetic resonance apparatus, in particular the scanner, following basic calibration it is possible to obtain permanent registration between the coordinate system of 3D camera and the coordinate system of the magnetic resonance apparatus so that position information in the scanning data can be converted directly into position information in the coordinate system of the magnetic resonance apparatus. However, it is also conceivable for the registration to be performed and updated using current scanning data. For this purpose, use can be made of the fact that the 3D camera also covers the patient table. Therefore, in an embodiment of the method, for the registration of the coordinate systems, at least one feature of the patient table, in particular a marker and/or a geometric feature, is localized in the scanning data by image processing and compared with the known position of the patient table. The possibility of three-dimensional scanning on the patient table itself can mean it is sufficient to scan only some features of the patient table, for example the corners thereof. It is also possible to arrange markers, which can be detected and localized in the scanning data, on and/or at the patient table. This is done using image processing algorithms as are known in the prior art for 3D cameras. In this way, the registration obtained is always highly accurate and fully up-to-date since the position approached is to control computer of the magnetic resonance apparatus.

Preferably, a movement of the patient table for the adaptation of the registration and/or during the determination of the at least one recording parameter can be taken into account and/or a receiving position of the patient table can be determined as a recording parameter. Since the adjustment of the patient table is usually controlled automatically by the control computer of the magnetic resonance apparatus and/or at least the position of the patient table can be tracked by the control computer of the magnetic resonance apparatus, it is therefore not detrimental for the scanning data to be recorded in the loading position since the movement of the patient on the patient table compared to the time of the recording of the scanning data can be tracked at any time and taken into account by a subsequent registration, during the determination of the recording parameters or also in some other way. It is also possible to use the evaluation of the scanning data to determine the optimal position of the patient table in the patient receiving region for the recording of the magnetic resonance data.

In a preferred embodiment of the present invention, the scanning data are evaluated for the determination or adaptation of a surface model of the patient. This means that the scanning data are used for the regeneration of a three-dimensional surface model or patient model or to adapt an existing patient model for the determination of the surface model. In this case, the second variant, with which a patient model is already present, from which an adapted instance is generated as a surface model, is preferred since it is then possible for background knowledge of the usual surfaces of humans can be included in the determination of the surface model, and the patient model can also be linked to more extensive information. For example, it is possible for the location of specific anatomical features to be described so that the position thereof can then also be determined within the (adapted) surface model. In this context, therefore, the patient model, which serves as a template, is coupled to an anatomical atlas or contains such an atlas. Such a surface model generally offers an extremely large number of advantageous possibilities for reading patient parameters that have a direct influence on the suitable setting of the at least one recording parameter.

For example, at least one extent and/or one volume and/or one weight and/or one body mass index (BMI) of the patient is determined as a patient parameter from the surface model of the patient, and the at least one patient parameter for the adaptation and/or selection of at least one of the at least one recording parameter is taken into account. For example, the extent of the patient in various directions can be used to calculate the degree of oversampling required to avoid wrapping artifacts. The weight or preferably the BMI of the patient enables the automated use of magnetic resonance sequences or magnetic resonance protocols optimized for fat or thin patients. Therefore, there is a large number of possibilities for determining the patient parameters characterizing the patient and for calculating optimal recording parameters automatically.

Moreover, it is preferable if, in the case of a registration between the coordinate system of the 3D camera and the coordinate system of the magnetic resonance apparatus, a position of the actual patient or a region of interest within the patient is determined and used for setting a recording parameter of the at least one recording parameter defining the receiving region. This is particularly useful when using a surface model of the patient using an anatomical atlas or registered with such an atlas. As soon as positions from the scanning data can also be transferred into the coordinate system of the magnetic resonance apparatus, they can be used for the automatic determination of position-related recording parameters. In the present case, the position can also contain the alignment, which can determine the alignment of gradient axes and the like, for example the phase-encoding direction. It is possible to determine the location of the region of interest in the patient, and correspondingly also automatically to determine recording parameters describing the receiving region, i.e. the field of view, such as the size thereof and location thereof.

A magnetic resonance examination may possibly entail a number of scans, i.e. a number of individual magnetic resonance sequences, for each of which patient-related recording parameters can be determined dependent on the scanning data. Therefore, the present invention also permits the definition of, for example, recording parameters for a so-called localizer scan, i.e. an overview scan before the actual recording of the diagnostic magnetic resonance data. In such a case, patient information obtained from the scanning data also can be transferred to the following, diagnostic scans, and the results of the localizer scan can be used to refine set recording parameters, particularly with respect to the region of interest of the patient.

As noted, the use of a 3D camera can simply and effectively be included in the workflow during a customary magnetic resonance examination since use is made of a process that would anyway be available and performed in order to determine additional information to automate or at least significantly simplify the setting of the recording parameters. This is because it is always necessary to position the patient on the patient table outside the patient receiving region, and the patient is usually initially not obscured by covering objects such as blankets and/or local coils. While all these activities can be performed by the operator, the scanning data can be recorded without any problem.

Therefore, in an embodiment of the present invention, at least during a preparation period for positioning the patient on the patient table, scanning data are continuously recorded, and upon the fulfillment of a positioning criterion, displaying a final positioning of the patient and evaluating the scanning data. Scanning data of the patient in the final positioning, showing the patient without a covering object that cannot be penetrated by the 3D camera, are automatically evaluated for the determination of the at least one recording parameter. For example, the 3D camera can always record scanning data when the patient table is moved out, in particular under the additional condition that information is available on an upcoming magnetic resonance examination. The scanning data are then evaluated dependent on a positioning criterion, so as to check whether the patient remains unmoved for a period of a predetermined duration and/or when the application of covering objects has started and/or whether a confirmation button for the final positioning of the patient is pressed. Further specific embodiments of the positioning criterion are also conceivable. In this case, it is not necessary for the determination of the recording parameters to use the scanning data recorded at or after the time of the fulfillment of the positioning criterion. Instead, it is possible to select the scanning data that show the patient covered as completely as possible and as little as possible in the same position/location. To this end, ideally, the scanning data are stored at least for a specified period, and temporal course of the scanning data is analyzed.

It is possible to continue the recording of the scanning data after the occurrence of the positioning criterion and to evaluate the scanning data obtained thereby with respect to the fulfillment of a repositioning criterion representing the repositioning of the patient, which is ideally fulfilled when at least one movement of the patient exceeding a threshold value has occurred. It is then possible, for example, to set an updating trigger with which at least a part of the recording parameters is determined again and/or updated. For example, recording parameters for positioning the patient can be determined again, with only recording parameters that are dependent upon unchanged patient parameters to be retained upon a repositioning.

In an embodiment of the method, the scanning data are also evaluated for the determination of at least one piece of coil information describing a local coil placed on the patient. Therefore, to improve the workflow, it is also possible at local coils for coil information that can be determined from scanning data to be collected and used for notifications or adaptations. For example, identification information and/or coil model information can be determined as the coil information, in particular by detection of at least one characterizing feature of the local coil in image processing, and/or coil position information is determined as the coil information. The coil information obtained in this way can be used to support the user and/or for improved determination of the recording parameters and/or further recording parameters. Accordingly, the coil information can also be evaluated for setting at least one of the at least one and/or one further recording parameter and/or with respect to an output of notifications to an operator. For example, notifications can be emitted if an incorrect local coil is used, a local coil is positioned incorrectly or unfavorably, and the like. It is also possible for coil information to be provided in another way, for example coil information obtained by inserting a coil plug into a slot, to be checked or validated.

The scanning data can also be evaluated for objects other than local coils. For example, it is possible for evaluation algorithms to be used that are able to detect and provide notification of disruptive objects that should not be placed in the patient receiving region, for example forgotten instruments and/or objects made of metal. In this case, a suitable warning notification can be emitted. Therefore, the use of a 3D camera with the magnetic resonance apparatus provides advantages that go beyond the determination of patient information for setting recording parameters.

The recording parameters do not necessarily have to be automatically set as invariable, but at least one determined recording parameter is offered as a variable preselection via a user interface. Experienced operators will then have the possibility of making modifications if this will enable improvements to be made on the basis of their experience. At the same time, however, the setting is made much easier for less experienced operators since it is simple for them to accept the recording parameters offered.

As noted, it is advantageous for the determination of the at least one recording parameter also to take account of at least one item of examination information that describes the examination to be performed, for example in order to identify the field of interest to be examined in the patient. Such examination information can be entered by an operator or accesses from a database, for example, a hospital information system (HIS) or a radiology information system (RIS). It is also possible for a patient record to be used as a source of such examination information.

In addition to the method, the invention also concerns a magnetic resonance apparatus having a data acquisition scanner that has a basic field magnet that defines a patient receiving region, a patient table that can be moved into the patient receiving region, a 3D camera that has a field of view that covers the patient table in an extended loading position, and a control computer that is programmed to implement the method according to the invention. The 3D camera is preferably arranged so as to be located centrally above the patient table in the loading position, and therefore is able to “see” the patient table (and a patient positioned thereupon) from above as completely as possible. All statements relating to the method according to the invention apply to the magnetic resonance apparatus according to the invention, with which it is therefore also possible to achieve the same advantages.

### BRIEF DESCRIPTION OF THE DRAWINGS

FIG. 1 schematically illustrates a magnetic resonance apparatus according to the invention.

FIG. 2 is a flowchart of an exemplary embodiment of the method according to the invention.

### DESCRIPTION OF THE PREFERRED EMBODIMENTS

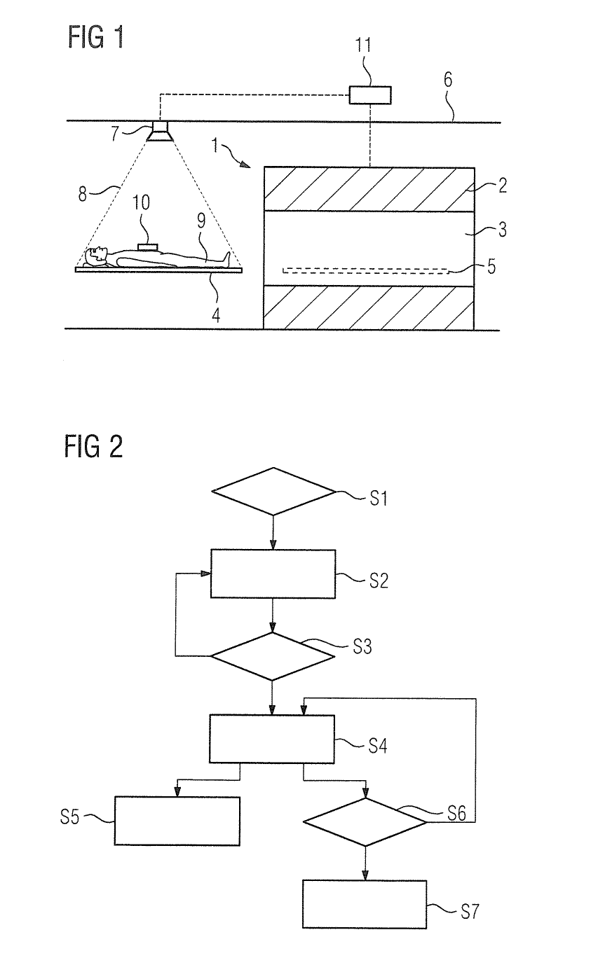

FIG. 1 is a schematic illustration of a scanner 1 of a magnetic resonance apparatus according to the invention. In FIG. 1, a section through the center of the scanner 1 is shown so that the patient receiving region 3 is also clearly identifiable. In this case, the scanner 1 has in addition to the basic field magnet 2, other customary components (not shown), such as a radio-frequency coil arrangement surrounding the patient receiving region 3 and a gradient coil arrangement surrounding the patient receiving region 3 and a cooling computer for the scanner 1, in addition to further customary components.

A patient support (not shown in further detail) is used to move a patient table 4, which in the present case is shown in a loading position outside the patient receiving region 3, into the patient receiving region 3, and back out. One possible position 5 within the patient receiving region 3 is indicated by dashed lines.

As can be seen, a 3D camera 7 is situated on the ceiling 6 of a room containing the scanner 1, centrally above the patient table 4 in the loading position. The field of view 8 of the 3D camera 7 permits a complete coverage of patient table 4 in the loading position and a patient 9 positioned on the patient table 4. Objects covering parts of the surface of the patient 9, such as local coil 10 to be placed thereupon, can also be detected in the three-dimensional scanning data of the 3D camera 7, which is here a time-of-flight camera.

The 3D camera 7 supplies scanning data acquired thereby to a control computer 11, which controls the operation of the entire magnetic resonance apparatus and which is also programmed to implement the method according to the invention.

FIG. 2 is a flowchart of an exemplary embodiment of the method according to the invention. In this case, ideally the 3D camera 7 is incorporated in the workflow of a magnetic resonance examination. In a step S1, a check is performed as to whether the patient table 4 is located in the loading position and whether a magnetic resonance examination is upcoming, as can be determined for example from examination information which can be entered by an operator and/or obtained from an information system. If this is the case, in a step S2 scanning data of the patient table 4 and objects located thereupon are recorded continuously. Unless there is already anyway a registration of the coordinate system of the 3D camera 7 and the coordinate system of the magnetic resonance scanner 1 to a fixed, known positioning of the 3D camera 7, registration can then take place in step S2 using the features of the patient table 4 obtained in the scanning data. In some exemplary embodiments, markers can be provided on the patient table 4, in order to simplify this process. The evaluation of features of the patient table 4 can be used to update an existing registration. The registration enables known movements of the patient table 4, controlled by the control computer 11, to be incorporated in all calculations containing scanning data, or based thereon.

In a step S3, a check is performed as to whether a positioning criterion for the patient 9 on the patient table 4 has occurred. This requires a patient 9 to be actually positioned on the patient table 4, which can be achieved by corresponding image processing measures and classification of the patient 9 in the scanning data. Also, there must be indications that the patient 9 is located in the patient's final position to be used for the recording of magnetic resonance data. For this purpose, it is possible for a corresponding actuating element to be used by the operator. It is also possible for this to be determined by image analysis of the scanning data, for example detecting that covering objects such as blankets/sheets and/or local coils 10 are arranged on the patient 9, and/or that the patient has not been moved for a long time.

If the positioning criterion has occurred, in a step S4, initially, scanning data are selected that are to be evaluated with respect to the surface of the patient 9. In this case, the scanning data ideally represent the surface of the patient 9 as completely as possible, which means as few as possible or no covering objects through which the 3D camera 7 is unable to scan are present. In this case, it is possible to use scan data that were recorded at a time before the occurrence of the positioning criterion, since the scanning data are retained for a predetermined period. Obviously scanning data should be selected with which the patient 9 was already in the position present at the time of the fulfillment of the positioning criterion.

The scanning data are subsequently evaluated. To this end, a model instance of a patient model, which is also provided with/or linked to an anatomical atlas, is adapted as a surface model to the surface of the patient 9 described by the scanning data so that, ideally, the outer surface thereof is completely described by the surface model. Then, patient parameters, in particular at least one extent and/or one volume and/or one weight and/or one body mass index of the patient are determined therefrom. Since, the field of interest within the patient 9 at which the upcoming magnetic resonance examination is directed is also known from the examination information, in addition to a position of the patient 9, a position or location of the region of interest within the patient 9 is also determined. The anatomical atlas is used for this purpose. This patient information, i.e. the patient parameters, and the positions are then used to determine patient-related recording parameters for the future recording of magnetic resonance data. For example, the BMI of the patient can be used as the basis for the selection of magnetic resonance sequences optimized for fat/thin patients, in this way an oversampling can be determined such that no wrapping artifacts occur, the receiving region can be defined with reference to the region of interest and/or it is possible for gradient directions, for example the phase-encoding direction, to be defined.

The recording parameters determined in this way are offered as a preselection in a step S5 at a user interface for setting recording parameters so that they can be easily accepted by an operator or, if required, changed once again. In other exemplary embodiments, it is also possible to automatically use the recording parameters directly.

In a step S6, the scanning data is further evaluated with respect to the fulfillment of a repositioning criterion, i.e. a subsequent change in the position of the patient 9, and with respect to other objects, which are represented by the scanning data and for which useful information can be derived in the workflow. If the repositioning criterion is fulfilled, the method branches back to step S4 with at least one part of the recording parameters being updated/re-determined in order to take account of the new position of the patient 9.

If it is desired to determine information on further objects, for example the local coil 10, the scanning data relative to these objects are evaluated in more detail in a step S7. Relative to the local coil 10, the detection of at least one characterizing feature of the local coil 10 in image processing, for example, enables the determination of identification information and/or coil model information as coil information. It is also possible to determine coil position information. This can also be taken into account when setting recording parameters; however, it is also possible for the coil information to be evaluated with respect to outputting information to an operator, for example if the incorrect local coil 10 is present and/or if said coil is positioned unfavorably. It is also possible for scanning data to be evaluated in respect of further objects, for example in order to ensure that no unwanted objects are moved into the patient receiving region 3 with the patient table 4 and the patient 9.

As soon as the patient table 4 is moved into the patient receiving region 3, the recording of scanning data with the 3D camera 7 is terminated.

Although modifications and changes may be suggested by those skilled in the art, it is the intention of the Applicant to embody within the patent warranted hereon all changes and modifications as reasonably and properly come within the scope of the Applicant's contribution to the art.

1. A method for operating a magnetic resonance (MR) apparatus, said MR apparatus comprising an MR data acquisition scanner and a patient table movable into a patient receiving region of the MR data acquisition scanner, said method comprising:
prior to operating the MR data acquisition scanner in order acquire MR data from a patient, placing the patient on the patient table at a loading position at which said patient table is adjacent to said MR data acquisition scanner, but not yet moved into said patient receiving region;
holding said patient table in said loading position and operating a 3D camera, having a field of view that covers the patient table in the loading position, to acquire three-dimensional scanning data representing a surface of the patient in the loading position on the patient table;
providing said scanning data to a processor and, in said processor, determining from said scanning data at least one patient-related recording parameter; and
emitting said patient-related recording parameter from said processor as an electronic signal in a form for use in subsequent operation of said MR data acquisition scanner to acquire MR data from the patient in the patient receiving region.

2. A method as claimed in claim 1 comprising positioning said 3D camera centrally above said patient table.

3. A method as claimed in claim 1 comprising selecting said 3D camera from the group consisting of time-of-flight cameras and terahertz cameras.

4. A method as claimed in claim 1 comprising, in said processor, prior to determining said at least one patient-related recording parameter, bringing a coordinate system of the 3D camera and a coordinate system of the MR data acquisition scanner into registration with each other.

5. A method as claimed in claim 4 comprising bringing said coordinate system of the 3D camera and the coordinate system of the MR data acquisition scanner into registration by acquiring, in said scanning data, at least one feature of the patient table that is recognizable in said scanning data and, in said processor, comparing a position of said at least one feature with a known position of said patient table.

6. A method as claimed in claim 4 comprising taking movement of the patient table into account when bringing said coordinate system of the 3D camera and the coordinate system of the MR data acquisition scanner into registration.

7. A method as claimed in claim 1 comprising taking movement of said patient table into account for determining said at least one patient-related recording parameter.

8. A method as claimed in claim 1 comprising determining a receiving position of the patient table as a recording parameter.

9. A method as claimed in claim 1 comprising evaluating said scanning data in said processor to determine or adapt a surface model of the patient.

10. A method as claimed in claim 9 comprising, from said surface model of the patient, determining at least one patient parameter selected from the group consisting of an extent of the patient in a selected direction, the volume of the patient, the weight of the patient, and the body mass index of the patient, and adapting or selecting said at least one patient-related recording parameter dependent on said at least one patient parameter.

11. A method as claimed in claim 9 comprising, in said processor, prior to determining said at least one patient-related recording parameter, bringing a coordinate system of the 3D camera and a coordinate system of the MR data acquisition scanner into registration with each other, and, from said registration, determining a position, selected from the group consisting of a position of the patient and a position of a region of interest within the patient, and using said position to determine said patient-related recording parameter as a recording parameter that defines said patient receiving region.

12. A method as claimed in claim 1 comprising, during a preparation period in which said patient is positioned on the patient table, continuously acquiring said scanning data with said 3D camera until fulfillment of a positioning criterion that indicates occurrence of a final positioning of the patient with no covering objects on the patient that cannot be penetrated by the 3D camera and, in said processor, automatically evaluating said final position of the patient for use in determining said at least one patient-related recording parameter.

13. A method as claimed in claim 1 comprising placing a local coil, to be used with said MR data acquisition scanner in order to acquire the MR data from the patient, on the patient on the patient table in said loading position, and operating said 3D camera so that said scanning data includes a representation of said local coil, and, in said processor, determining at least one item of coil information describing said local coil from said scanning data.

14. A method as claimed in claim 12 comprising detecting said at least one item of coil information from the group consisting of coil identification information and coil model information, by detecting at least one characterizing feature of said local coil by image processing.

15. A method as claimed in claim 12 comprising detecting coil position information as said at least one item of coil information.

16. A method as claimed in claim 10 comprising, in said processor, evaluating said at least one item of coil information in order to set at least one additional recording parameter for operating said MR data acquisition scanner in order to acquire said MR data from the patient.

17. A method as claimed in claim 10 comprising using said at least one item of coil information to generate a notification that designates an error associated with said local coil, and emitting said notification from said processor at a user interface in communication with said processor.

18. A method as claimed in claim 1 comprising providing said at least one patient-related recording parameter at a user interface in communication with said processor, as a variable pre-selection available to an operator of the MR data acquisition scanner.

19. A method as claimed in claim 1 comprising providing said processor with at least one item of examination information that describes an examination to be performed with respect to said patient in said MR data acquisition scanner, and determining said at least one patient-related recording parameter dependent on said at least one item of examination information.

20. A magnetic resonance (MR) apparatus, comprising:
an MR data acquisition scanner and a patient table movable into a patient receiving region of the MR data acquisition scanner;
prior to operating the MR data acquisition scanner in order acquire MR data from a patient, the patient table being moved to a loading position at which said patient table is adjacent to said MR data acquisition scanner, but not yet moved into said patient receiving region, with a patient being placed on the patient table at said loading position, and said patient table being held in said loading position;
a 3D camera, having a field of view that covers the patient table in the loading position, configured to acquire three-dimensional scanning data representing a surface of the patient in the loading position on the patient table;
a processor provided with said scanning data, said processor being configured to determine from said scanning data, at least one patient-related recording parameter; and
said processor being configured to emit said patient-related recording parameter from said processor as an electronic signal in a form for use in subsequent operation of said MR data acquisition scanner to acquire MR data from the patient in the patient receiving region.
